(19) United States
(12) Patent Application Publication
(10) Pub. No.: US 2019/0262483 A1
(43) Pub. Date: Aug. 29, 2019

(54) Sterilization Assembly and Related Methods

(71) Applicant: Bausch Advanced Technologies, Inc., Clinton, CT (US)

(72) Inventors: Eugen Goidenko, Ivoryton, CT (US); Markus Rink, Branford, CT (US); Pavel Sergeyevich Grigorash, New London, CT (US)

(73) Assignee: Bausch Advanced Technologies, Inc., Clinton, CT (US)

(21) Appl. No.: 16/288,963

(22) Filed: Feb. 28, 2019

Related U.S. Application Data
(60) Provisional application No. 62/636,386, filed on Feb. 28, 2018.

(51) Int. Cl. A61L 2/00; A61L 2/26

(52) U.S. Cl. CPC A61L 2/0047; A61L 2/26; A61L 2202/122; A61L 2202/21; A61L 2202/11; A61L 2202/182; A61L 2202/121

(57) ABSTRACT

The present disclosure relates to sterilization assemblies and related methods and, more particularly, to assemblies and methods for ultraviolet (“UV”) light sterilization of items (e.g., container members and/or packaging, such as packaged containers that house products) for subsequent introduction of the sterilized items to a desired environment (e.g., to an ISO rated cleanroom environment for processing pharmaceutical products; to an isolator environment positioned in a cleanroom for processing pharmaceutical products). The present disclosure provides for improved assemblies/methods for sterilizing items for subsequent introduction of the sterilized items to a desired environment. The assemblies/methods of the present disclosure advantageously allow an end user to quickly, effectively, safely, cost-effectively and/or easily sterilize items for subsequent introduction to a desired environment, thereby substantially eliminating drawbacks of conventional designs/systems, and also providing significant operational, manufacturing and/or commercial advantages as a result.

### CROSS-REFERENCE TO RELATED APPLICATIONS

This application claims the benefit of U.S. Provisional Application entitled “STERILIZATION ASSEMBLY AND RELATED METHODS,” which was filed on Feb. 28, 2018, and assigned Ser. No. 62/636,386, the contents of which are herein incorporated by reference in their entirety.

### FIELD OF THE DISCLOSURE

The present disclosure relates to sterilization assemblies and related methods and, more particularly, to assemblies and methods for ultraviolet (“UV”) light sterilization of items (e.g., container members and/or packaging) for subsequent introduction of the sterilized items to a desired environment (e.g., to an ISO rated cleanroom environment for processing pharmaceutical products; to an isolator positioned for processing pharmaceutical products).

### BACKGROUND OF THE DISCLOSURE

In general, some systems and methods for attempting to sterilize packaging and/or items for subsequent introduction to a cleanroom environment (e.g., a cleanroom environment for processing pharmaceutical products) are known.

For example, vaporized hydrogen peroxide (“VHP”) systems and methods have been utilized to attempt to sterilize items for subsequent introduction to a desired environment. However, such VHP systems and methods can be dangerous, large, expensive, slow and/or time-consuming (e.g., lasting at least two or three hours to attempt to sterilize).

Other sterilization attempts include spraying and/or wiping the items of interest with cleaning chemicals or the like, waiting for an extended period of time, and then introducing the sprayed/wiped items to the desired environment. However, such systems and methods (e.g., “spray-and-pray” systems/methods) can be ineffective (e.g., leave unsterilized areas), non-repeatable, slow and/or time-consuming (e.g., lasting at least one or two hours to attempt to sterilize).

Thus, an interest exists for improved assemblies/methods for sterilizing items for subsequent introduction of the sterilized items to a desired environment. These and other inefficiencies and opportunities for improvement are addressed and/or overcome by the assemblies, systems and methods of the present disclosure.

### SUMMARY OF THE DISCLOSURE

The present disclosure provides advantageous sterilization assemblies, and improved methods for using the same. More particularly, the present disclosure provides improved assemblies and methods for UV light sterilization of items (e.g., container members and/or packaging, such as packaged containers that house products) for subsequent introduction of the sterilized items to a desired environment (e.g., to an ISO rated cleanroom environment for processing pharmaceutical products; to an isolator positioned in a cleanroom for processing pharmaceutical products).

In general, the present disclosure provides for improved assemblies/methods for sterilizing items for subsequent introduction of the sterilized items to a desired environment. As such, the assemblies/methods of the present disclosure advantageously allow an end user to quickly, effectively, safely, cost-effectively and/or easily sterilize items for subsequent introduction to a desired environment, thereby substantially eliminating drawbacks of conventional designs/systems, and also providing significant operational, manufacturing and/or commercial advantages as a result.

The present disclosure provides for a sterilization assembly including a housing defining an internal chamber, the internal chamber including a shelf member configured to support a container member; a first door member positioned on a first side of the housing, the first door member configured to allow access to the internal chamber from the first side of the housing; a second door member positioned on a second side of the housing, the second door member configured to allow access to the internal chamber from the second side of the housing; and a first light source mounted with respect to the housing, the first light source configured and adapted to supply light to the internal chamber to sterilize the container member.

The present disclosure also provides for a sterilization assembly wherein the first light source is an ultraviolet light source; and wherein the first light source is configured and adapted to provide ultraviolet light for about thirty seconds or less to the internal chamber to sterilize the container member.

The present disclosure also provides for a sterilization assembly wherein the first light source is configured and adapted to provide light in the UV-C to near infrared wavelengths to the internal chamber to sterilize the container member.

The present disclosure also provides for a sterilization assembly wherein the shelf member is transparent to UV light; and wherein the first light source supplies light to the internal chamber to sterilize the container member after the first and second door members are closed.

The present disclosure also provides for a sterilization assembly further including a barrier sealed to at least portions of the housing, the barrier separating a first environment from a second environment; and wherein the first door member is positioned adjacent to the first environment, and the second door member is positioned adjacent to the second environment.

The present disclosure also provides for a sterilization assembly wherein the first environment is an unsterilized environment, and the second environment is a sterilized environment. The present disclosure also provides for a sterilization assembly wherein the sterilized environment is an ISO rated cleanroom environment or an isolator environment for processing pharmaceutical products.

The present disclosure also provides for a sterilization assembly wherein the container member houses one or more pre-sterilized medical products.

The present disclosure also provides for a sterilization assembly further including a second light source and a third light source mounted with respect to the housing, the second and third light sources configured and adapted to supply light to the internal chamber to sterilize the container member. The present disclosure also provides for a sterilization assembly wherein the housing includes a top side, a bottom side, a first end and a second end; and wherein the bottom side of the housing includes the first light source positioned adjacent to the internal chamber, and the top side of the housing includes the second and third light sources positioned adjacent to the internal chamber.

The present disclosure also provides for a sterilization assembly wherein the housing includes at least one reflector member mounted with respect to the internal chamber. The present disclosure also provides for a sterilization assembly wherein each reflector member is a diffuse reflector member, and each reflector member is configured and dimensioned to be about 95% to about 98% reflective.

The present disclosure also provides for a sterilization method including providing a housing defining an internal chamber, the internal chamber including a shelf member; positioning a first door member on a first side of the housing, the first door member configured to allow access to the internal chamber from the first side of the housing; positioning a second door member on a second side of the housing, the second door member configured to allow access to the internal chamber from the second side of the housing; mounting a first light source with respect to the housing; closing the first and second door members; opening the first door member and positioning a container member on the shelf member from the first side of the housing; closing the first door member; supplying light to the internal chamber, via the first light source, to sterilize the container member; and opening the second door member and removing the container member from the internal chamber from the second side of the housing.

The present disclosure also provides for a sterilization method wherein the first light source is configured and adapted to provide ultraviolet light for about thirty seconds or less to the internal chamber to sterilize the container member.

The present disclosure also provides for a sterilization method further including a barrier sealed to at least portions of the housing, the barrier separating a first environment from a second environment; and wherein the first door member is positioned adjacent to the first environment, and the second door member is positioned adjacent to the second environment.

The present disclosure also provides for a sterilization method wherein the first environment is an unsterilized environment, and the second environment is a sterilized environment. The present disclosure also provides for a sterilization method wherein the sterilized environment is an ISO rated cleanroom environment or an isolator environment for processing pharmaceutical products.

The present disclosure also provides for a sterilization method wherein the container member houses one or more pre-sterilized medical products.

The present disclosure also provides for a sterilization method wherein after opening the first door member and positioning the container member on the shelf member, the second door member can be opened and the sterilized container member can be removed from the internal chamber in about 30 seconds or less.

The present disclosure also provides for a sterilization assembly including a housing defining an internal chamber, the internal chamber including a transparent shelf member configured to support a container member; a barrier sealed to at least portions of the housing, the barrier separating a first environment from a second environment; a first door member positioned on a first side of the housing, the first door member configured to allow access to the internal chamber from the first side of the housing; a second door member positioned on a second side of the housing, the second door member configured to allow access to the internal chamber from the second side of the housing; and a first UV light source mounted with respect to the housing, the first UV light source configured and adapted to supply UV light to the internal chamber to sterilize an outer packaging layer of the container member; wherein the first UV light source is configured and adapted to provide ultraviolet light for about thirty seconds or less to the internal chamber to sterilize the outer packaging layer of the container member; wherein the first door member is positioned adjacent to the first environment, and the second door member is positioned adjacent to the second environment; wherein the first environment is an unsterilized environment, and the second environment is a sterilized environment; wherein the sterilized environment is an ISO rated cleanroom environment or an isolator environment for processing pharmaceutical products; and wherein the container member houses one or more pre-sterilized medical products.

Any combination or permutation of embodiments is envisioned. Additional advantageous features, functions and applications of the disclosed assemblies, methods and systems of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

Features and aspects of embodiments are described below with reference to the accompanying drawings, in which elements are not necessarily depicted to scale.

Exemplary embodiments of the present disclosure are further described with reference to the appended figures. It is to be noted that the various features, steps and combinations of features/steps described below and illustrated in the figures can be arranged and organized differently to result in embodiments which are still within the scope of the present disclosure. To assist those of ordinary skill in the art in making and using the disclosed assemblies, systems and methods, reference is made to the appended figures, wherein:

FIG. 1 is a side perspective view of a sterilization assembly according to an exemplary embodiment of the present disclosure;

FIG. 2 is a side view of the sterilization assembly of FIG. 1;

FIG. 3 is another side view of the sterilization assembly of FIG. 1;

FIG. 4 is a cross-sectional side view of the sterilization assembly of FIG. 3;

FIGS. 5-11 are partial side perspective views of the sterilization assembly of FIG. 1;

FIG. 12 is a top view of a sterilization assembly positioned in an environment for processing pharmaceutical products, according to an exemplary embodiment of the present disclosure;

FIG. 13 is a partial side perspective view of another sterilization assembly according to an exemplary embodiment of the present disclosure; and

FIGS. 14-15 are partial side perspective views of the sterilization assembly of FIG. 13.

### DETAILED DESCRIPTION OF DISCLOSURE

The exemplary embodiments disclosed herein are illustrative of advantageous sterilization assemblies of the present disclosure and methods/techniques thereof. It should be understood, however, that the disclosed embodiments are merely exemplary of the present disclosure, which may be embodied in various forms. Therefore, details disclosed herein with reference to exemplary sterilization assemblies/fabrication methods and associated processes/techniques of assembly and use are not to be interpreted as limiting, but merely as the basis for teaching one skilled in the art how to make and use the advantageous sterilization assemblies and/or alternative assemblies of the present disclosure.

In general, the present disclosure provides improved sterilization assemblies, and related methods of use. More particularly, the present disclosure provides advantageous assemblies and methods for UV light sterilization of items (e.g., container members and/or packaging, such as packaged containers that house products) for subsequent introduction of the sterilized items to a desired environment (e.g., to an ISO rated cleanroom environment, such as, for example, an ISO 5 cleanroom, for processing pharmaceutical products; to an isolator positioned in a cleanroom for processing pharmaceutical products).

Current practice provides that some attempted sterilization systems/methods (e.g., VHP systems) can be dangerous, large, expensive, slow and/or time-consuming (e.g., lasting at least two or three hours to attempt to sterilize), and other systems/methods (“spray-and-pray” systems) can be ineffective (e.g., leave unsterilized areas), non-repeatable, slow and/or time-consuming (e.g., lasting at least one or two hours to attempt to sterilize).

In exemplary embodiments, the present disclosure provides for improved assemblies/methods for sterilizing items for subsequent introduction of the sterilized items to a desired environment. For example, the assemblies/methods of the present disclosure advantageously allow an end user to quickly (e.g., in about 30 seconds or less), effectively, safely, cost-effectively and/or easily sterilize items for subsequent introduction to a desired environment. As such, the noted improvements of the assemblies/methods of the present disclosure thereby substantially eliminate drawbacks of conventional designs/systems, and also provide significant operational, manufacturing and/or commercial advantages as a result.

In general, some UV sterilization systems are known. Some exemplary systems in this general field are described and disclosed in U.S. Pat. Nos. 5,786,598 and 8,125,333; and U.S. Patent Pub. Nos. 2009/0274576 and 2011/0155915, the entire contents of each being hereby incorporated by reference in their entireties.

Referring now to the drawings, like parts are marked throughout the specification and drawings with the same reference numerals, respectively. Drawing figures are not necessarily to scale and in certain views, parts may have been exaggerated for purposes of clarity.

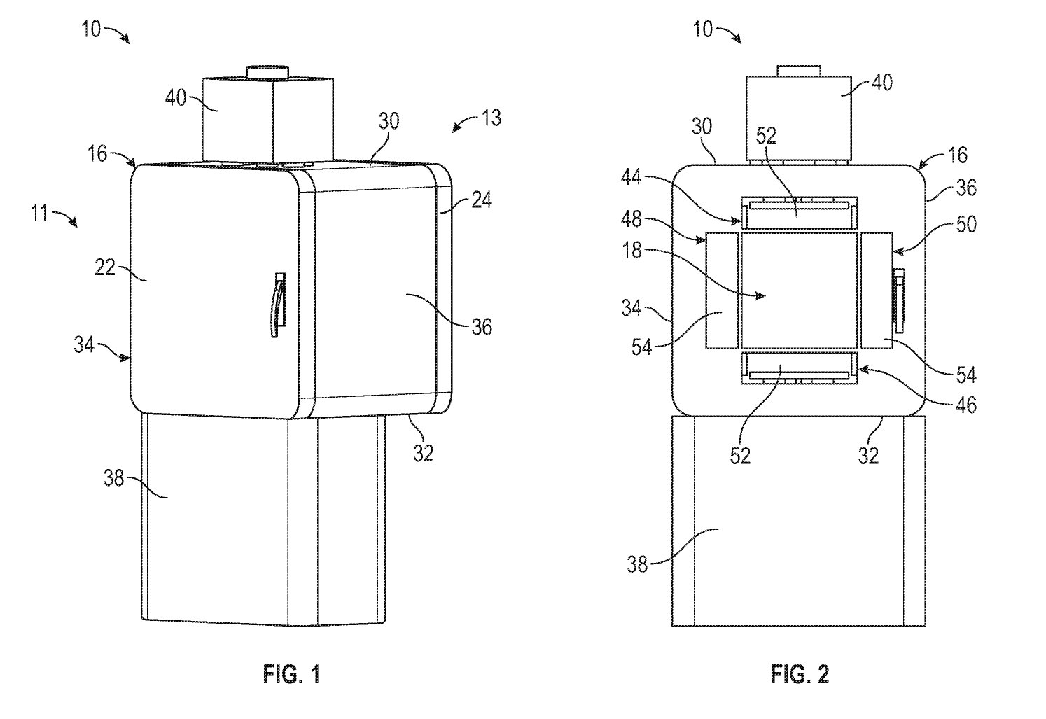

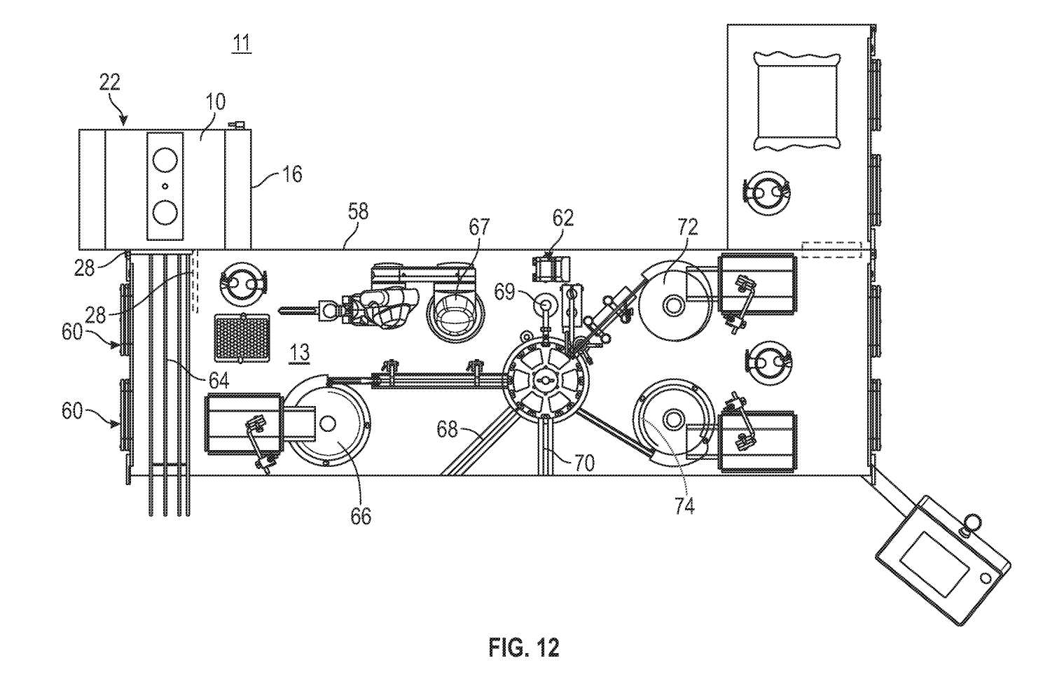

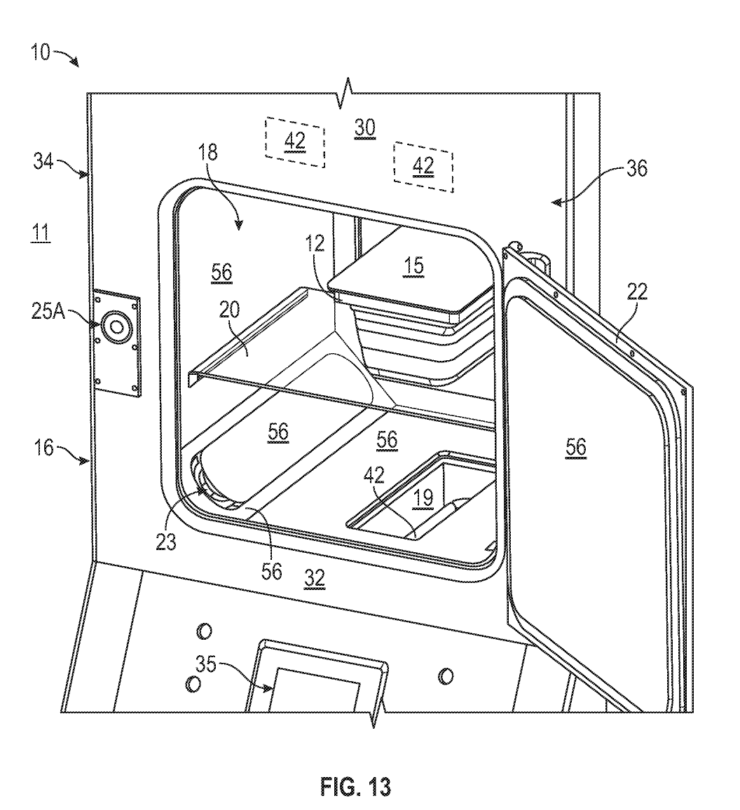

With reference to FIGS. 1-4, there is illustrated an embodiment of an exemplary sterilization assembly 10 according to the present disclosure. In general, sterilization assembly 10 is configured and dimensioned to sterilize items 12 (e.g., via UV light) for subsequent introduction of the sterilized items 12 to a desired environment 13 (FIGS. 4 and 12). As discussed further below, it is noted that sterilization assembly 10 can take a variety of forms, shapes, sizes and/or designs.

Exemplary sterilization assembly 10 can be configured to sterilize, via UV light, items 12 (e.g., container members 12 and/or packaging 12, such as packaged containers 12 that house one or more products 14) for subsequent introduction of the sterilized items 12 to a desired environment 13 (e.g., to an ISO rated cleanroom environment 13 for processing pharmaceutical products; to an isolator environment 13 positioned for processing pharmaceutical products).

For example and as discussed further below, item 12 can be located in a first environment 11 (e.g., unsterilized environment 11). The item 12 may be positioned in the sterilization assembly 10, sterilized by assembly 10, and then the sterilized item 12 may be introduced/delivered to the desired environment 13 (e.g., to an ISO 1 through ISO 9 classified cleanroom environment 13, etc., or to any other desired cleanroom or sterilized environment 13 for processing pharmaceutical products or the like).

In other example embodiments and as discussed further below, the sterilized item 12 may be introduced/delivered to an isolator environment 13 positioned in a room or the like (e.g., to an isolator environment 13 positioned in a cleanroom), the isolator environment 13 mounted to or associated with processing equipment/machinery or the like (e.g., equipment/machinery for processing pharmaceutical products, such as, for example, a filling machine).

Exemplary item 12 takes the form of a container member 12 or the like, although the present disclosure is not limited thereto. Rather, it is noted that item 12 can take a variety of shapes, forms and/or designs.

In exemplary embodiments, item 12 is configured to house and/or contain one or more products 14 (e.g., a plurality of pre-sterilized products 14, such as, for example, pre-sterilized medical devices 14—syringes 14, bottles 14, IV bags 14, etc.). In some embodiments, it is noted that item 12 can include an outer packaging layer 15 or the like.

As such, item 12 can be located in a first environment 11 (e.g., unsterilized environment 11), the item 12 can then be positioned in the sterilization assembly 10, then sterilized by assembly 10 (e.g., to sterilize an outer packaging layer 15 of item 12), and then the sterilized item 12 may be introduced/delivered to the desired environment 13. The products 14 can then be removed from sterilized item 12 in the desired environment 13 for further processing (e.g., in conjunction with equipment/machinery for processing pharmaceutical products, such as, for example, a filling machine or the like).

With reference now to FIGS. 1-4, exemplary sterilization assembly 10 includes a housing 16 that defines an internal chamber 18 therein (e.g., a two-foot by two-foot, substantially square internal chamber 18). It is noted that internal chamber 18 can take a variety of other shapes, forms and/or designs.

Exemplary internal chamber 18 includes a shelf member 20, the shelf member 20 configured and dimensioned to support, hold and/or house the item 12 within internal chamber 18 for sterilization purposes. Exemplary shelf member 20 takes the form of a quartz shelf member 20 or the like (e.g., UV light transparent quartz shelf member 20). In general, shelf member 20 is transparent to UV light, and can be fabricated from other suitable materials.

Exemplary housing 16 includes a first outer door member 22 and a second outer door member 24. In some embodiments, housing 16 includes first inner door member 26 and second inner door member 28. It is noted that housing 16 can include any combination or permutation of door members 22, 24, 26 and 26, as long as there is at least one door on the side adjacent to environment 11 and at least one door on the side adjacent to environment 13 (e.g., housing 16 could include doors 22, 26 and 28, and not include door 24, etc.).

In general, when door members 22, 24, 26 and 28 are in the closed position (FIG. 4), the internal chamber 18 is closed and forms an isolated UV sterilization chamber 18. It is noted that in embodiments where door members 26, 28 are not present, when door members 22 and 24 are in the closed position, then the internal chamber 18 is closed and forms an isolated UV sterilization chamber 18. Likewise, in embodiments where door members 22, 24 are not present, when door members 26 and 28 are in the closed position, then the internal chamber 18 is closed and forms an isolated UV sterilization chamber 18.

Exemplary housing 16 also includes a top side 30, a bottom side 32, a first end 34 and a second end 36. An electrical box 38 or the like can be mounted to the housing 16 (e.g., to bottom side 32), and one or more exhaust units 40 can be mounted to housing 16 (e.g., to top side 30, for light sources 42).

In exemplary embodiments, assembly 10 includes one or more light sources 42 (e.g., UV light sources 42) mounted with respect to the housing 16, each light source 42 configured to provide or supply sterilizing light to the internal chamber 18 to sterilize the item 12 (e.g., to sterilize an outer packaging layer 15 of item 12) positioned in chamber 18 (e.g., to supply sterilizing light to the item 12 positioned on shelf 20 in the internal chamber 18 to sterilize an outer packaging layer 15 of item 12).

In exemplary embodiments, the top side 30 of housing 16 includes a first mounting cavity 44 positioned adjacent to chamber 18, and bottom side 32 of housing 16 includes a second mounting cavity 46 positioned adjacent to chamber 18.

Similarly, the first end 34 of housing 16 includes a third mounting cavity 48 positioned adjacent to chamber 18, and the second end 36 of housing 16 includes a fourth mounting cavity 50 positioned adjacent to chamber 18.

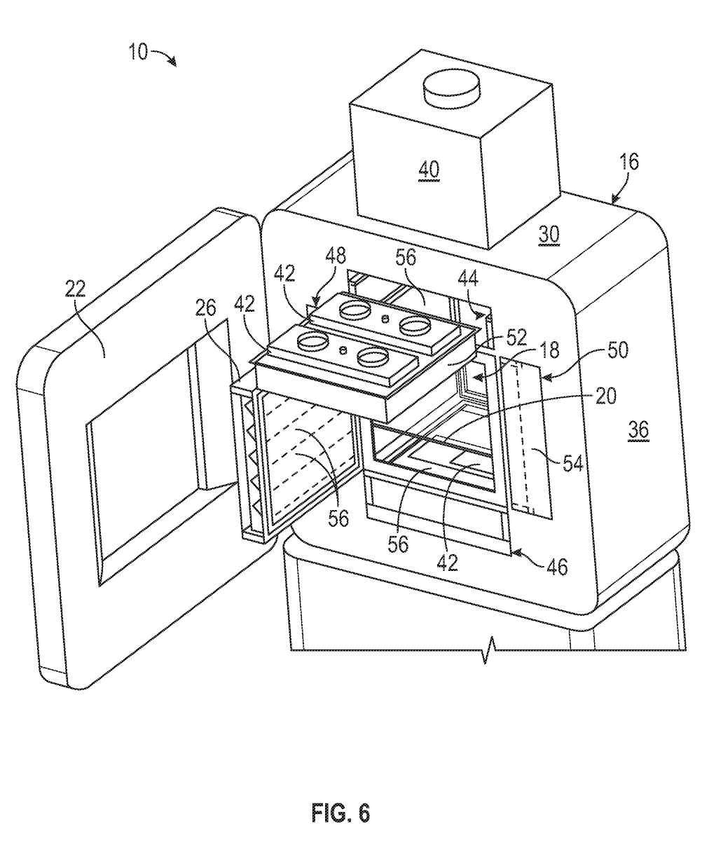

In general, each mounting cavity 44, 46, 48, 50 is configured and dimensioned to releasably house and/or secure a lighting sub-assembly 52 or a reflector sub-assembly 54 positioned adjacent to internal chamber 18. As shown in FIG. 6, each exemplary lighting sub-assembly 52 or reflector sub-assembly 54 is removable or replaceable from cavities 44, 46, 48, 50 (e.g., for maintenance, repair, interchangeability, replacement, etc.). In certain embodiments, it is noted that access to lighting sub-assembly 52 or reflector sub-assembly 54 in cavities 44, 46, 48, 50 can be provided on the side of housing 16 adjacent to environment 11 (e.g., when door 22 is opened or removed), and that access to lighting sub-assembly 52 or reflector sub-assembly 54 in cavities 44, 46, 48, 50 is not provided on the side of housing 16 adjacent to environment 13. Each cavity 44, 46, 48, 50 can be covered by an access panel (e.g., hinged access panel) or the like for covering each cavity 44, 46, 48, 50 when not being accessed by a user.

For example and as shown in FIG. 2, the first and second mounting cavities 44, 46 each can be configured and dimensioned to releasably house and/or secure a lighting sub-assembly 52 positioned adjacent to internal chamber 18, and the third and fourth mounting cavities 48, 50 each can be configured and dimensioned to releasably house and/or secure a reflector sub-assembly 54 positioned adjacent to internal chamber 18.

In general, each lighting sub-assembly 52 can house or contain one or more light sources 42 (e.g., UV light sources 42). It is noted that each lighting sub-assembly 52 can also house or contain one or more reflector members 56 positioned adjacent to the light sources 42 of the sub-assembly, and positioned adjacent to internal chamber 18. In other words and in certain embodiments, the internal surface of each lighting sub-assembly 52 positioned adjacent to chamber 18 is either a light source 42 surface or a reflector member 56 surface.

Similarly, each reflector sub-assembly 54 can house or contain one or more reflector members 56 positioned adjacent to internal chamber 18. As such and in certain embodiments, the internal surface of each reflector sub-assembly 54 positioned adjacent to chamber 18 is a reflector member 56 surface.

In certain embodiments and as shown in FIG. 2, the first and second mounting cavities 44, 46 each removably secure a lighting sub-assembly 52 positioned adjacent to internal chamber 18, and the third and fourth mounting cavities 48, 50 each removably secure a reflector sub-assembly 54 positioned adjacent to internal chamber 18. In this example embodiment, the lighting sub-assembly 52 in the first mounting cavity 44 includes two light sources 42 (e.g., UV light sources 42), and the lighting sub-assembly 52 in the second mounting cavity 46 includes one light source 42 (FIG. 4). However, it is to be noted that other permutations or combinations of light sources 42 in cavities 44, 46 is contemplated (e.g., one, three, a plurality, etc. of light sources 42 in cavity 44; two, three, a plurality, etc. of light sources 42 in cavity 46).

Likewise, it is also to be noted that any other suitable combination or permutation of lighting sub-assemblies 52 and/or reflector sub-assemblies 54 in cavities 44, 46, 48, 50 is contemplated (e.g., lighting sub-assemblies 52 in cavities 48, 50 and reflector sub-assemblies 54 in cavities 44, 46; one lighting sub-assembly 52 in cavity 44 and reflector sub-assemblies 54 in cavities 46, 48, 50; one reflector sub-assembly 54 in cavity 44 and lighting sub-assemblies 52 in cavities 46, 48, 50; etc., with other suitable combinations/permutations of sub-assemblies 52, 54 in cavities 44, 46, 48, 50).

In general, each light source 42 (e.g., UV light source 42) is configured and adapted to provide or supply sterilizing light to the internal chamber 18 to sterilize the item 12 (e.g., to sterilize an outer packaging layer 15 of item 12) positioned in chamber 18. For example, each light source 42 can provide or supply light (e.g., pulses of UV light) for sterilization purposes of item 12 (e.g., provide UV germicidal irradiation (UVGI) that uses short-wavelength ultraviolet (UV-C) light to kill or inactivate microorganisms). In exemplary embodiments, each light source 42 provides or supplies light (e.g., sustained UV light or multiple pulses of UV light over a time span of about thirty seconds or less to chamber 18) in the UV-C to near infrared wavelengths for sterilization purposes of item 12 (e.g., to sterilize an outer packaging layer 15 of item 12).

For example, each light source 42 (e.g., Xenon light source 42) can have an output of about 3 Hz to about 100 Hz, and can deliver sustained UV light or multiple pulses of UV light (e.g., each pulse being about 3 ms in duration) over the desired time span (e.g., a time span of about three seconds; a time span of about ten seconds; a time span of about fifteen seconds; a time span of about thirty seconds; etc.).

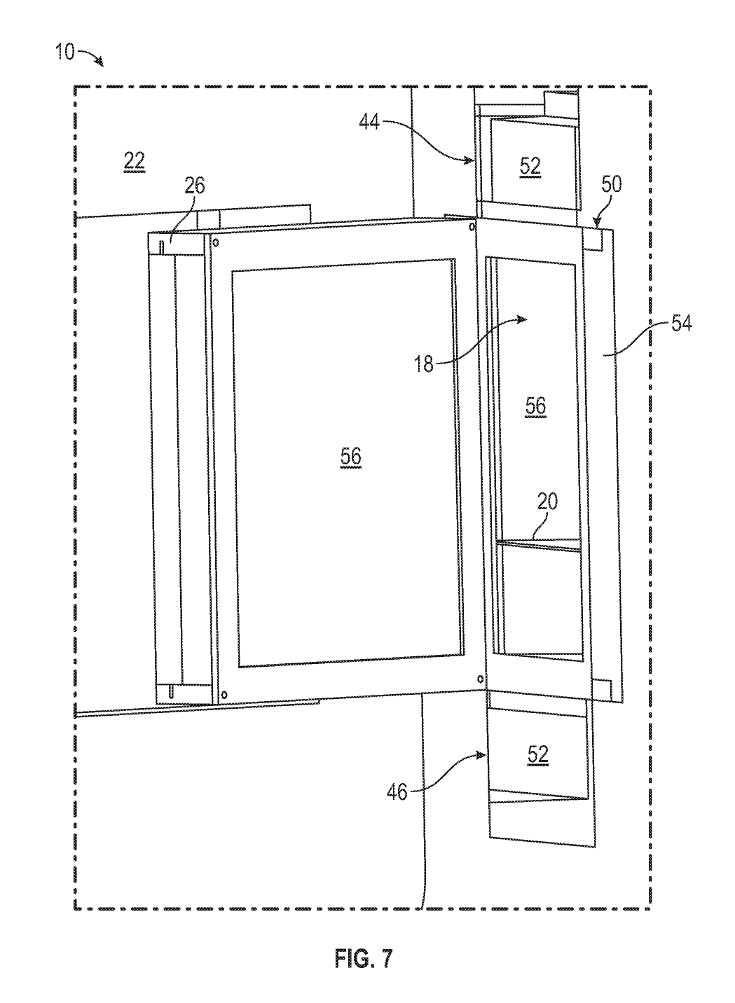

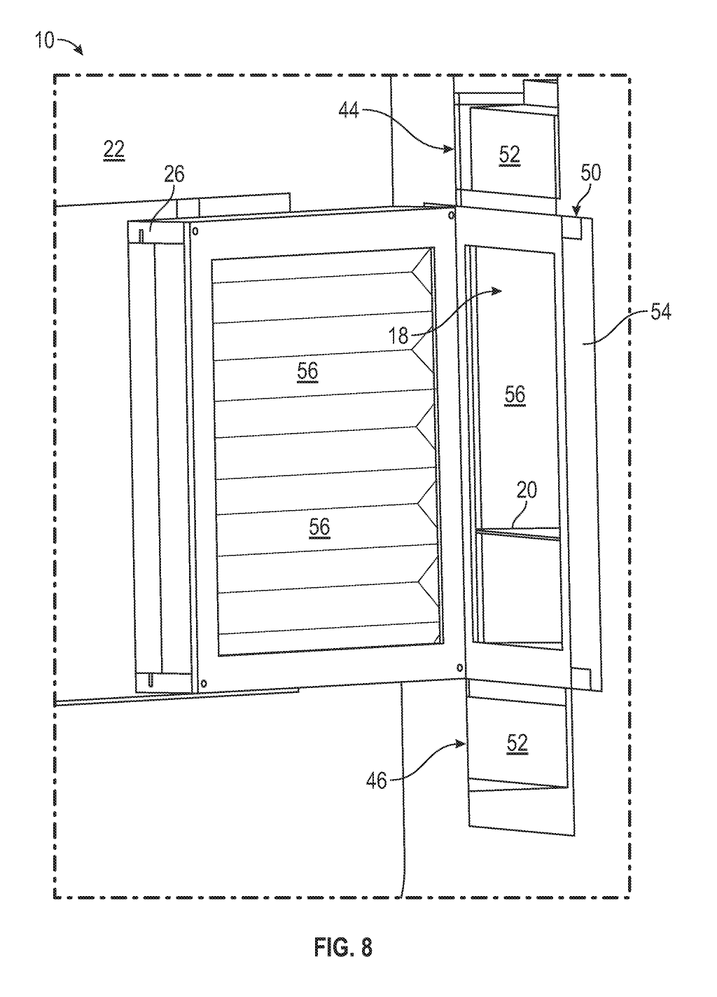

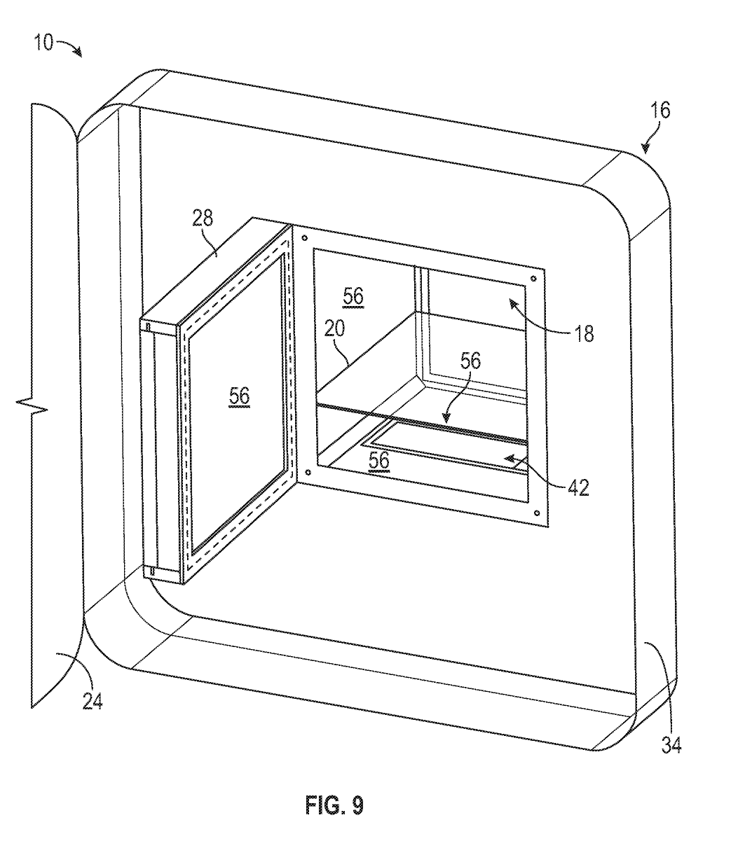

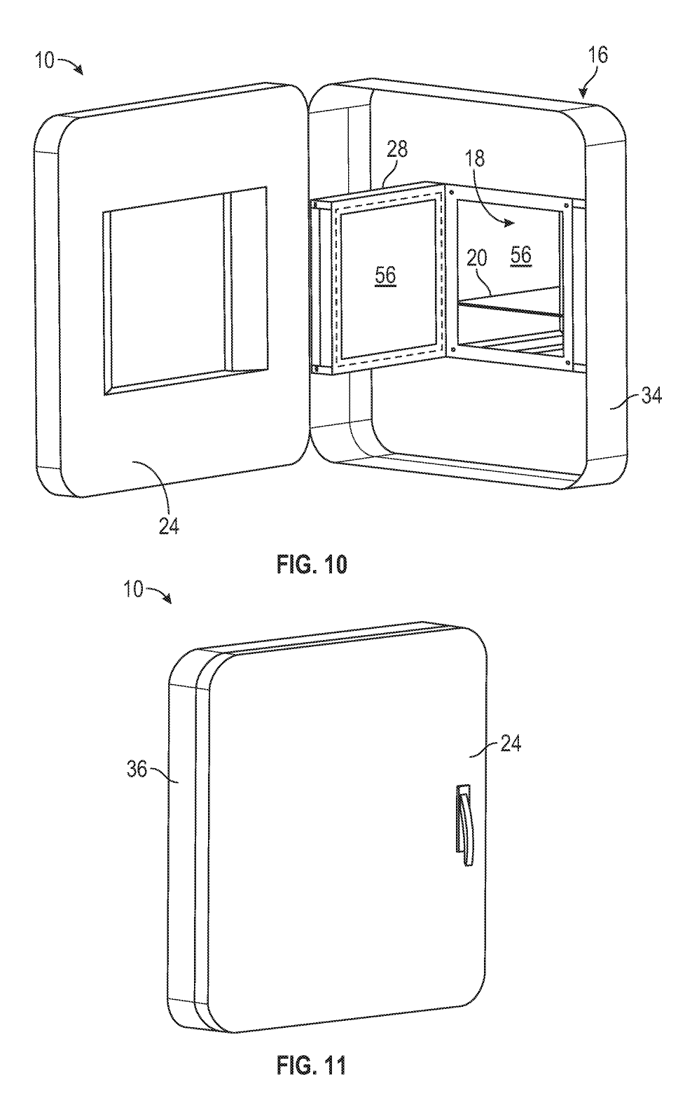

In exemplary embodiments, each reflector member 56 is a diffuse reflector member 56 or the like, and each reflector member 56 is configured and dimensioned to be about 95% to about 98% reflective. Exemplary reflector members 56 can be fabricated from polytetrafluoroethylene (PTFE) or the like. Reflector members 56 can be substantially flat or planar (FIG. 7), or can be angled (FIG. 8). Other shapes, designs and forms of reflector members 56 can also be utilized.

It is noted that the inner surfaces of inner door members 26, 28 can include one or more reflector members 56 or the like. As such and in certain embodiments, when the item 12 is positioned on the shelf member 20, and the lighting sub-assemblies 52 and/or reflector sub-assemblies 54 are releasably positioned in cavities 44, 46, 48, 50, and when the inner door members 26, 28 are in the closed position (FIG. 4), and then when the light sources 42 deliver light to the chamber 18 for sterilization purposes, it is noted that substantially every surface surrounding the internal chamber 18 is either light reflective (e.g., via reflector members 56) or light transparent (e.g., shelf 20) or is a light source (e.g., light sources 42), except for item 12 positioned in chamber 18 to be sterilized.

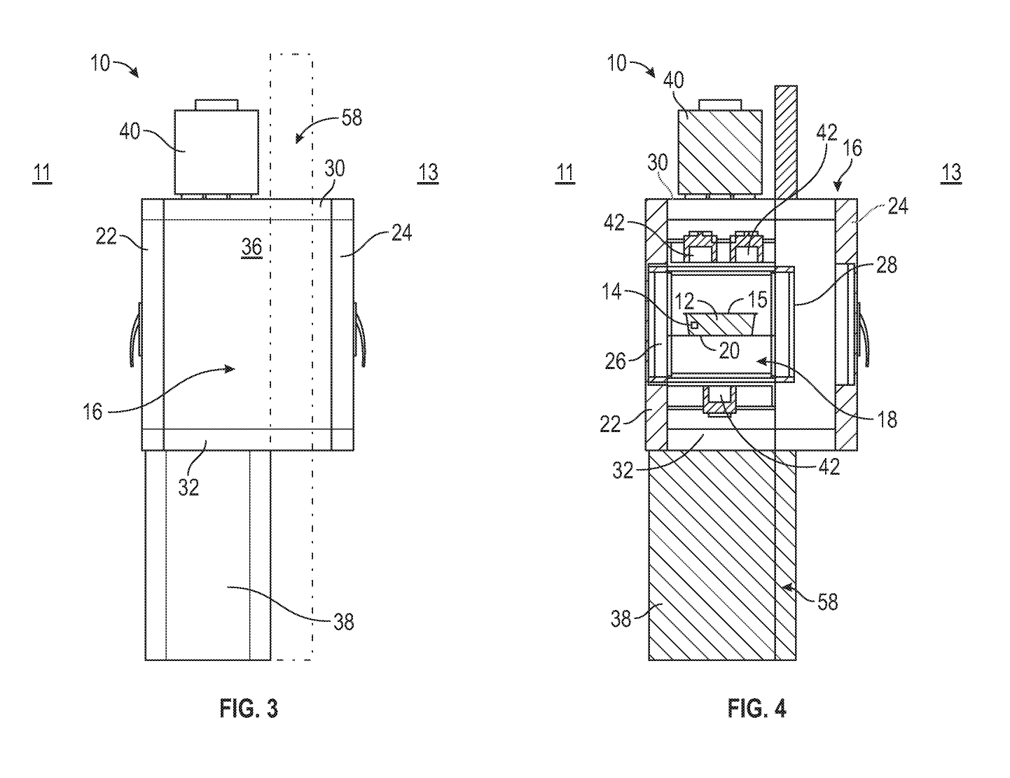

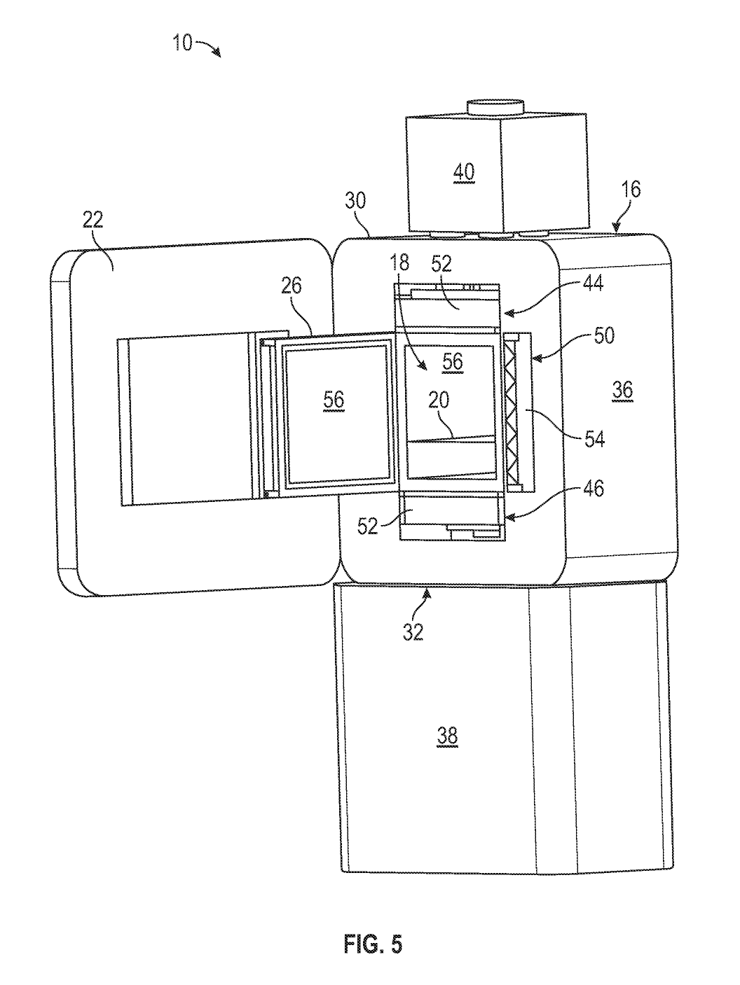

In certain embodiments and as shown in FIGS. 3-4, at least portions of the housing 16 of sterilization assembly 10 can be mounted with respect to a wall or barrier 58 of a desired environment 13 (e.g., cleanroom environment 13). For example, at least portions of the outer surfaces of the housing 16 can be sealed (e.g., air-tight sealed) to wall/barrier 58. Thus and as shown in FIGS. 3-4, the environment 11 on the left side of the wall/barrier 58 can be an unsterilized environment 11, and the environment 13 on the right side of the wall/barrier 58 can be a sterilized environment 13.

For example, item 12 can be located in the first environment 11 (e.g., unsterilized environment 11). The item 12 may be positioned in the sterilization assembly 10, sterilized by assembly 10 (e.g., to sterilize an outer packaging layer 15 of item 12), and then the sterilized item 12 may be introduced/delivered to the second, desired environment 13 (e.g., to an ISO classified cleanroom environment 13, or to any other desired cleanroom environment 13 for processing pharmaceutical products or the like).

More particularly, the item 12 may be first positioned in the first environment 11, and the door members 22, 24, 26 and 28 of the housing 16 are all in the closed position (FIG. 4). A first user in environment 11 may then open first outer door member 22 and first inner door member 26, and place the item 12 in internal chamber 18. Door members 22 and 26 are then put in the closed position, and then the item 12 can be sterilized by the one or more light sources 42 that provide or supply light (e.g., pulses of UV light for about three seconds or less to chamber 18) for sterilization purposes of item 12 (e.g., to sterilize an outer packaging layer 15 of item 12).

After item 12 is sterilized (e.g., to sterilize an outer packaging layer 15 of item 12), another user in second environment 13 can then open second outer door member 24 and second inner door member 28, and then retrieve item 12 from chamber 18 and introduce sterilized item 12 to environment 13 for processing purposes or the like. Doors 24 and 28 can then be closed.

For example, after sterilized item 12 is introduced to environment 13, a user may open the sterilized item 12 and retrieve the one or more products 14 (e.g., a plurality of pre-sterilized products 14, such as, for example, pre-sterilized medical devices 14—syringes 14, bottles 14, IV bags 14, etc.) contained in item 12 for processing purposes (e.g., in conjunction with equipment/machinery for processing pharmaceutical products, such as, for example, a filling machine or the like).

It is noted that such an exemplary process/method of putting item 12 in chamber 18 from environment 11, sterilizing item 12 via light sources 42, and introducing item 12 to environment 13 can take about 60 seconds or less in some embodiments, and can take about 30 seconds or less in other embodiments.

In certain embodiments, it is noted that item 12 can include an outer packaging layer 15 or the like. In such embodiments, it is noted that the sterilization of item 12 via light sources 42 supplying light to chamber 18 includes the sterilization of the outer packaging layer 15 or the like of item 12.

It is further noted that the assembly 10 can include (optional) features and/or means for ensuring that the surface of the outer packaging layer 15 of the item 12 (or the surface of the item 12 itself, if there is no layer 15) is substantially evened out or stretched/straightened out prior to sterilization via light sources 42. For example, after item 12 is positioned in chamber 18 and doors 22, 24, 26 and 28 are closed, the chamber 18 can be in communication with one or more devices (e.g., vacuum pump and blower; vacuuming features/means; features/means for changing the pressure in chamber 18) that can reduce the pressure in the chamber 18, thereby substantially evening out or stretching/straightening out the surface of item 12 prior to sterilization of item 12 via light sources 42.

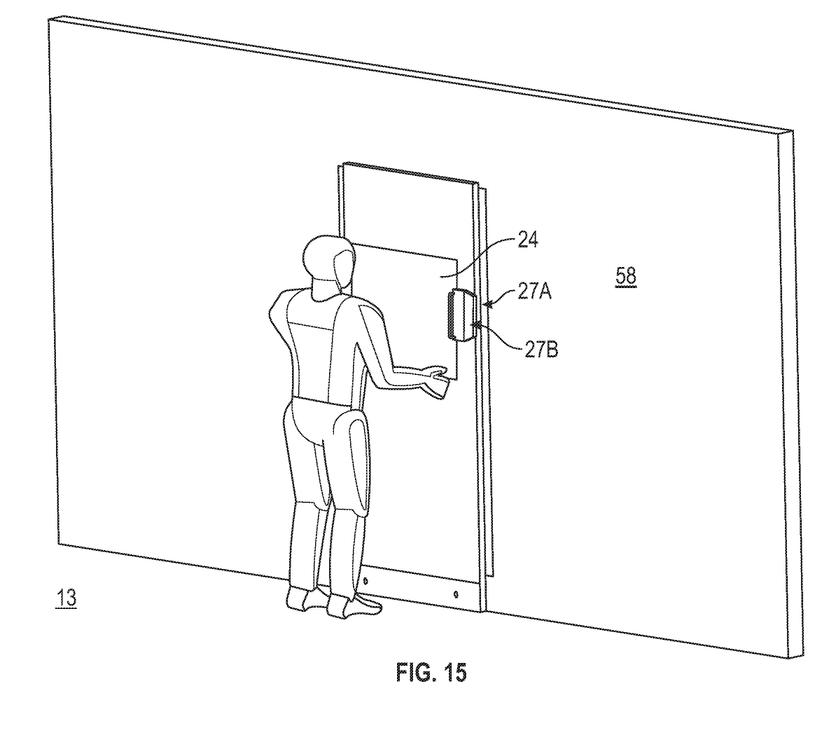

In another embodiment and as depicted in FIGS. 13-15, sterilization assembly 10 is configured and dimensioned to sterilize items 12 (e.g., to sterilize an outer packaging layer 15 of items 12) for subsequent introduction of the sterilized items 12 to a desired environment 13.

Sterilization assembly 10 includes a housing 16 that defines an internal chamber 18 therein, with internal chamber 18 including a shelf member 20 (e.g., quartz shelf member 20), the shelf member 20 configured and dimensioned to support, hold and/or house the item 12 within internal chamber 18 for sterilization purposes.

Exemplary housing 16 includes a first outer door member 22 and a second outer door member 24. In general, when door members 22, 24 are in the closed position, the internal chamber 18 is closed and forms an isolated UV sterilization chamber 18.

Exemplary housing 16 also includes a top side 30, a bottom side 32, a first end 34 and a second end 36. One or more exhaust units 40 can be mounted to housing 16 (e.g., to top side 30, for light sources 42).

Assembly 10 includes one or more light sources 42 (e.g., UV light sources 42) mounted with respect to the housing 16, each light source 42 configured to provide or supply sterilizing light to the internal chamber 18 to sterilize the item 12 (e.g., to sterilize an outer packaging layer 15 of item 12) positioned in chamber 18 (e.g., to supply sterilizing light to the item 12 positioned on shelf 20 in the internal chamber 18 to sterilize an outer packaging layer 15 of item 12).

In exemplary embodiments, the bottom side 32 of housing 16 includes a first light source 42 positioned adjacent to chamber 18, and top side 30 of housing 16 includes a second light source 42 and a third light source 42 positioned adjacent to chamber 18. In general, each light source is covered by a window member 19 or the like, to allow light to travel from the light source 42 to the internal chamber 18.

It is noted that bottom side 32 and/or top side of housing 16 can include any suitable number of light sources 42 positioned adjacent to chamber 18 (e.g., none, one, two, three, a plurality, etc.). Similarly, it is noted that first end 34 and/or second end 36 of housing 16 can include any suitable number of light sources 42 positioned adjacent to chamber 18 (e.g., none, one, two, three, a plurality, etc.).

In exemplary embodiments, the internal surface of chamber 18 is either a light source 42 surface (e.g., via window member 19) or a reflector member 56 surface.

In general, each light source 42 (e.g., UV light source 42) is configured and adapted to provide or supply sterilizing light to the internal chamber 18 to sterilize the item 12 (e.g., to sterilize an outer packaging layer 15 of item 12) positioned in chamber 18. For example, each light source 42 can provide or supply light (e.g., pulses of UV light) for sterilization purposes of item 12 (e.g., provide UV germicidal irradiation (UVGI) that uses short-wavelength ultraviolet (UV-C) light to kill or inactivate microorganisms). In exemplary embodiments, each light source 42 provides or supplies light (e.g., sustained UV light or multiple pulses of UV light over a time span of about thirty seconds or less to chamber 18) in the UV-C to near infrared wavelengths for sterilization purposes of item 12 (e.g., to sterilize an outer packaging layer 15 of item 12).

For example, each light source 42 (e.g., Xenon light source 42) can have an output of about 3 Hz to about 100 Hz, and can deliver sustained UV light or multiple pulses of UV light (e.g., each pulse being about 3 ms in duration) over the desired time span (e.g., a time span of about three seconds; a time span of about ten seconds; a time span of about fifteen seconds; a time span of about thirty seconds; etc.).

In general, each reflector member 56 is a diffuse reflector member 56 or the like, and each reflector member 56 is configured and dimensioned to be about 95% to about 98% reflective. Exemplary reflector members 56 can be fabricated from polytetrafluoroethylene (PTFE) or the like.

In certain embodiments, substantially the entire chamber 18 (except window members 19) includes a modular reflector member 56 (e.g., that is fabricated from polytetrafluoroethylene (PTFE) or the like) that substantially covers the entire interior of internal chamber 18. In general, such reflector members 56 are modular to accommodate different light source 42 set-up variations within chamber 18.

It is noted that the inner surfaces of door members 22, 24 can include one or more reflector members 56 or the like. As such and in certain embodiments, when the item 12 is positioned on the shelf member 20, and when the door members 22, 24 are in the closed position, and then when the light sources 42 deliver light to the chamber 18 for sterilization purposes, it is noted that substantially every surface surrounding the internal chamber 18 is either light reflective (e.g., via reflector members 56) or light transparent (e.g., shelf 20) or is a light source (e.g., light sources 42 via window members 19), except for item 12 positioned in chamber 18 to be sterilized.

In general, at least portions of the housing 16 of sterilization assembly 10 can be mounted with respect to a wall or barrier 58 of a desired environment 13 (e.g., cleanroom environment 13). For example, at least portions of the outer surfaces of the housing 16 can be sealed (e.g., air-tight sealed) to wall/barrier 58. Thus, the environment 11 can be an unsterilized environment 11, and the environment 13 can be a sterilized environment 13.

For example, item 12 can be located in the first environment 11 (e.g., unsterilized environment 11). The item 12 may be positioned in the sterilization assembly 10, sterilized by assembly 10 (e.g., to sterilize an outer packaging layer 15 of item 12), and then the sterilized item 12 may be introduced/delivered to the second, desired environment 13 (e.g., to an ISO classified cleanroom environment 13, or to any other desired cleanroom environment 13 for processing pharmaceutical products or the like).

More particularly, the item 12 may be first positioned in the first environment 11, and the door members 22, 24 of the housing 16 are all in the closed position. A first user in environment 11 may then open first outer door member 22, and place the item 12 in internal chamber 18. Door member 22 is then put in the closed position, and then the item 12 can be sterilized by the one or more light sources 42 that provide or supply light (e.g., pulses of UV light for about three seconds or less to chamber 18) for sterilization purposes of item 12 (e.g., to sterilize an outer packaging layer 15 of item 12).

After item 12 is sterilized (e.g., to sterilize an outer packaging layer 15 of item 12), another user in second environment 13 can then open second outer door member 24, and then retrieve item 12 from chamber 18 and introduce sterilized item 12 to environment 13 for processing purposes or the like.

For example, after sterilized item 12 is introduced to environment 13, a user may open the sterilized item 12 and retrieve the one or more products 14 (e.g., a plurality of pre-sterilized products 14, such as, for example, pre-sterilized medical devices 14—syringes 14, bottles 14, IV bags 14, etc.) contained in item 12 for processing purposes (e.g., in conjunction with equipment/machinery for processing pharmaceutical products, such as, for example, a filling machine or the like).

It is noted that such an exemplary process/method of putting item 12 in chamber 18 from environment 11, sterilizing item 12 via light sources 42, and introducing item 12 to environment 13 can take about 60 seconds or less in some embodiments, and can take about 30 seconds or less in other embodiments.

In certain embodiments, it is noted that item 12 can include an outer packaging layer 15 or the like. In such embodiments, it is noted that the sterilization of item 12 via light sources 42 supplying light to chamber 18 includes the sterilization of the outer packaging layer 15 or the like of item 12.

It is further noted that the assembly 10 can include (optional) features and/or means for ensuring that the surface of the outer packaging layer 15 of the item 12 (or the surface of the item 12 itself, if there is no layer 15) is substantially evened out or stretched/straightened out prior to sterilization via light sources 42.

For example and as shown in FIG. 13, after item 12 is positioned in chamber 18 and doors 22, 24 are closed, the chamber 18 can be in communication with one or more vacuum devices 23 (e.g., vacuum pump and blower; vacuuming features/means; features/means for changing the pressure in chamber 18) that can reduce the pressure in the chamber 18, thereby substantially evening out or stretching/straightening out the surface of item 12 prior to sterilization of item 12 via light sources 42.

Moreover and as shown in FIGS. 13-15, the environment 11 side of housing 16 can include a first interlock member 25A, and the door member 22 can include a second interlock member 25B.

Similarly, the environment 13 side of housing 16 can include a third interlock member 27A, and the door member 24 can include a second interlock member 27B.

In exemplary embodiments, first and second interlock members 25A, 25B are configured to magnetically and/or electrically interlock with one another when door member 22 is closed, and third and fourth interlock members 27A, 27B are configured to magnetically and/or electrically interlock with one another when door member 24 is closed.

As such and when both door members 22, 24 are closed, such interlocking features of members 25A, 25B, 27A, 27B will only allow either door member 22 or door member 24 to open at that time. Stated another way, the interlocking features of members 25A, 25B, 27A, 27B prevent both door members 22, 24 from being open simultaneously.

For example, if door members 22, 24 are both closed and then door member 22 is opened by a user, then interlock members 27A, 27B will not allow door member 24 to be opened while door member 22 remains open.

Similarly, if door members 22, 24 are both closed and then door member 24 is opened by a user, then interlock members 25A, 25B will not allow door member 22 to be opened while door member 24 remains open.

Thus, the interlocking features of members 25A, 25B, 27A, 27B prevent both door members 22, 24 from being open simultaneously. This is important for mitigating risk of exposing the clean environment 13 to the ambient (non-clean) atmosphere/environment 11.

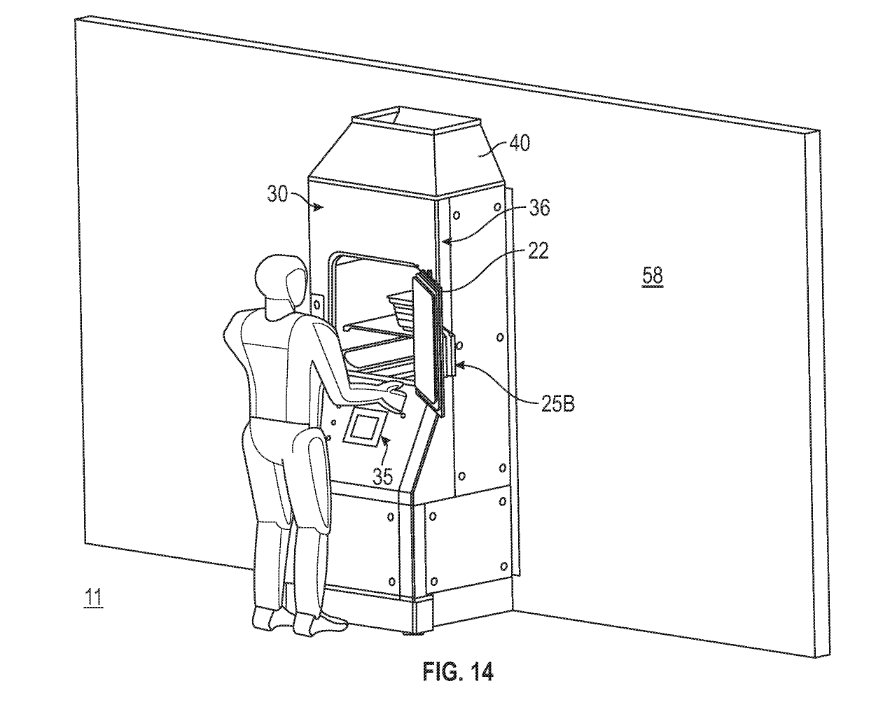

As shown in FIGS. 13 and 14, sterilization assembly 10 can also include a control panel 35 (e.g., human-machine interface control panel 35, mounted with respect to housing 16), for use by a user to control and/or monitor sterilization assembly 10.

In other embodiments and as shown in FIG. 12, at least portions of the housing 16 of sterilization assembly 10 can be mounted with respect to a wall or barrier 58 of a desired environment 13 (e.g., to an isolator environment 13 positioned for processing pharmaceutical products). For example, at least portions of the outer surfaces of the housing 16 can be sealed (e.g., air-tight sealed) to wall/barrier 58 of isolator environment 13. Thus and as shown in FIG. 12, the environment 11 outside the wall/barrier 58 can be an unsterilized environment 11, and the environment 13 inside the wall/barrier 58 can be a sterilized environment 13.

For example, item 12 can be located in the first environment 11 (e.g., unsterilized environment 11). The item 12 may be positioned in the sterilization assembly 10, sterilized by assembly 10 (e.g., to sterilize an outer packaging layer 15 of item 12), and then the sterilized item 12 may be introduced/delivered to the second, desired environment 13 (e.g., to the isolator environment 13 positioned for processing pharmaceutical products).

More particularly, the item 12 may be first positioned in the first environment 11, and the door members 22, 26 and 28 of the housing 16 are all in the closed position. In this embodiment, it is noted that housing 16 may not include second outer door member 24.

A first user in environment 11 may then open first outer door member 22 and first inner door member 26, and place the item 12 in internal chamber 18. Door members 22 and 26 are then put in the closed position, and then the item 12 can be sterilized by the one or more light sources 42 that provide or supply light (e.g., pulses of UV light for about three seconds or less to chamber 18) for sterilization purposes of item 12 (e.g., to sterilize an outer packaging layer 15 of item 12).

After item 12 is sterilized (e.g., to sterilize an outer packaging layer 15 of item 12), another user or robot member in second environment 13 can then open second inner door member 28 (e.g. via user ports 60—the dashed-line door 28 in FIG. 12 shows the open position), and then retrieve item 12 from chamber 18 and introduce sterilized item 12 to environment 13 for processing purposes or the like. Door 28 can then be closed.

For example, after sterilized item 12 is introduced to environment 13, a user or robot member may open the sterilized item 12 and retrieve the one or more products 14 (e.g., a plurality of pre-sterilized products 14, such as, for example, pre-sterilized medical devices 14—syringes 14, bottles 14, IV bags 14, etc.) contained in item 12 for processing purposes (e.g., in conjunction with a filling machine 62 or the like).

Exemplary filling machine 62 can include de-lidding area 64, sorting bowl 66, de-nesting robot 67, discharge area 68, filling station 69, reject station 70, plunger insertion area 72 and lock placement area 74, although the present disclosure is not limited thereto. Rather, it is noted that isolator environment 13 can take a variety of forms and designs.

It is noted that such an exemplary process/method of putting item 12 in chamber 18 from environment 11, sterilizing item 12 via light sources 42, and introducing item 12 to isolator environment 13 can take about 60 seconds or less in some embodiments, and can take about 30 seconds or less in other embodiments.

As such, the present disclosure provides for improved assemblies 10 and related methods for sterilizing items 12 (e.g., to sterilize an outer packaging layer 15 of items 12) for subsequent introduction of the sterilized items 12 to a desired environment 13. Thus, the assemblies 10 and related methods of the present disclosure advantageously allow an end user to quickly (e.g., in 30 seconds or less), effectively, safely, cost-effectively and/or easily sterilize items 12 for subsequent introduction to a desired environment 13. As such, the noted improvements of the assemblies 10 and methods of the present disclosure thereby substantially eliminate drawbacks of conventional designs/systems, and also provide significant operational, manufacturing and/or commercial advantages as a result.

Although the systems/methods of the present disclosure have been described with reference to exemplary embodiments thereof, the present disclosure is not limited to such exemplary embodiments/implementations. Rather, the systems/methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. A sterilization assembly comprising:
a housing defining an internal chamber, the internal chamber including a shelf member configured to support a container member;
a first door member positioned on a first side of the housing, the first door member configured to allow access to the internal chamber from the first side of the housing;
a second door member positioned on a second side of the housing, the second door member configured to allow access to the internal chamber from the second side of the housing; and
a first light source mounted with respect to the housing, the first light source configured and adapted to supply light to the internal chamber to sterilize the container member.

2. The sterilization assembly of claim 1, wherein the first light source is an ultraviolet light source; and
wherein the first light source is configured and adapted to provide ultraviolet light for about thirty seconds or less to the internal chamber to sterilize the container member.

3. The sterilization assembly of claim 1, wherein the first light source is configured and adapted to provide light in the UV-C to near infrared wavelengths to the internal chamber to sterilize the container member.

4. The sterilization assembly of claim 1, wherein the shelf member is transparent to UV light; and
wherein the first light source supplies light to the internal chamber to sterilize the container member after the first and second door members are closed.

5. The sterilization assembly of claim 1 further comprising a barrier sealed to at least portions of the housing, the barrier separating a first environment from a second environment; and
wherein the first door member is positioned adjacent to the first environment, and the second door member is positioned adjacent to the second environment.

6. The sterilization assembly of claim 5, wherein the first environment is an unsterilized environment, and the second environment is a sterilized environment.

7. The sterilization assembly of claim 6, wherein the sterilized environment is an ISO rated cleanroom environment or an isolator environment for processing pharmaceutical products.

8. The sterilization assembly of claim 1, wherein the container member houses one or more pre-sterilized medical products.

9. The sterilization assembly of claim 1 further comprising a second light source and a third light source mounted with respect to the housing, the second and third light sources configured and adapted to supply light to the internal chamber to sterilize the container member.

10. The sterilization assembly of claim 9, wherein the housing includes a top side, a bottom side, a first end and a second end;
wherein the bottom side of the housing includes the first light source positioned adjacent to the internal chamber, and the top side of the housing includes the second and third light sources positioned adjacent to the internal chamber.

11. The sterilization assembly of claim 1, wherein the housing includes at least one reflector member mounted with respect to the internal chamber.

12. The sterilization assembly of claim 11, wherein each reflector member is a diffuse reflector member, and each reflector member is configured and dimensioned to be about 95% to about 98% reflective.

13. A sterilization method comprising:
providing a housing defining an internal chamber, the internal chamber including a shelf member;
positioning a first door member on a first side of the housing, the first door member configured to allow access to the internal chamber from the first side of the housing;
positioning a second door member on a second side of the housing, the second door member configured to allow access to the internal chamber from the second side of the housing;
mounting a first light source with respect to the housing;
closing the first and second door members;
opening the first door member and positioning a container member on the shelf member from the first side of the housing;
closing the first door member;
supplying light to the internal chamber, via the first light source, to sterilize the container member; and
opening the second door member and removing the container member from the internal chamber from the second side of the housing.

14. The sterilization method of claim 13, wherein the first light source is configured and adapted to provide ultraviolet light for about thirty seconds or less to the internal chamber to sterilize the container member.

15. The sterilization method of claim 13 further comprising a barrier sealed to at least portions of the housing, the barrier separating a first environment from a second environment; and
wherein the first door member is positioned adjacent to the first environment, and the second door member is positioned adjacent to the second environment.

16. The sterilization method of claim 15, wherein the first environment is an unsterilized environment, and the second environment is a sterilized environment.

17. The sterilization method of claim 16, wherein the sterilized environment is an ISO rated cleanroom environment or an isolator environment for processing pharmaceutical products.

18. The sterilization method of claim 13, wherein the container member houses one or more pre-sterilized medical products.

19. The sterilization method of claim 13, wherein after opening the first door member and positioning the container member on the shelf member, the second door member can be opened and the sterilized container member can be removed from the internal chamber in about 30 seconds or less.

20. A sterilization assembly comprising:
a housing defining an internal chamber, the internal chamber including a transparent shelf member configured to support a container member;
a barrier sealed to at least portions of the housing, the barrier separating a first environment from a second environment;
a first door member positioned on a first side of the housing, the first door member configured to allow access to the internal chamber from the first side of the housing;
a second door member positioned on a second side of the housing, the second door member configured to allow access to the internal chamber from the second side of the housing; and
a first UV light source mounted with respect to the housing, the first UV light source configured and adapted to supply UV light to the internal chamber to sterilize an outer packaging layer of the container member;
wherein the first UV light source is configured and adapted to provide ultraviolet light for about thirty seconds or less to the internal chamber to sterilize the outer packaging layer of the container member;
wherein the first door member is positioned adjacent to the first environment, and the second door member is positioned adjacent to the second environment;
wherein the first environment is an unsterilized environment, and the second environment is a sterilized environment;
wherein the sterilized environment is an ISO rated cleanroom environment or an isolator environment for processing pharmaceutical products; and
wherein the container member houses one or more pre-sterilized medical products.
